(19) United States
(12) Patent Application Publication
(10) Pub. No.: US 2026/0350995 A1
(43) Pub. Date: Nov. 5, 2026

(54) PATIENT OCCUPANCY DETECTION USING 3D PASSIVE STEREO CAMERA AND POINT CLOUD PROCESSING USING AI/ML ALGORITHMS

(71) Applicant: KONINKLIJKE PHILIPS N.V., EINDHOVEN (NL)

(72) Inventors: GHOUSE MOHIDEEN BABU, BANGALORE (IN); MADHU JAHAGIRDAR, BOTHELL, MA (US); FALK UHLEMANN, NORDERSTEDT (DE); SIVA CHAITANYA CHADUVULA, MALDEN, MA (US); EKIN KOKER, CAMBRIDGE, MA (US); OLGA STAROBINETS, NEWTON, MA (US)

(21) Appl. No.: 19/112,518

(22) PCT Filed: Sep. 14, 2023
(86) PCT No.: PCT/EP2023/075222

§ 371 Date: Mar. 17, 2025

Related U.S. Application Data
(60) Provisional application No. 63/408,503, filed on Sep. 21, 2022.

(51) Int. Cl. G06V 20/52; G06V 10/70; G06V 10/72; G06V 40/10; G06V 40/20

(52) U.S. Cl. CPC G06V 20/52; G06V 10/70; G06V 10/72; G06V 40/10; G06V 40/20

(57) ABSTRACT

The present invention relates to patient occupancy detection. An apparatus is provided for processing data acquired from an area in a healthcare facility, for transfer to a cloud-based processing system for processing at the cloud-based processing system, comprising an input unit, a point cloud data generator, and an output unit. The input unit is configured to receive the data acquired from the area in the healthcare facility that is to be sent to the cloud-based processing system, comprising one or more of video data, image data, and ranging data. The point cloud data generator is configured to process the acquired data to generate point cloud data usable to provide a point cloud rendering of the area in the healthcare facility. The output unit is configured to communicate with a network to transfer the generated point cloud data and corresponding metadata to the cloud-based processing system.

### FIELD OF THE INVENTION

The present invention relates to patient occupancy detection, and in particular relates to an apparatus and a method for processing data acquired from an area in a healthcare facility, for transfer to a cloud-based processing system for processing at the cloud-based processing system, to a cloud-based processing system, to a system, to a computer program product, and to a computer-readable medium.

### BACKGROUND OF THE INVENTION

In a multi-vendor and multi-modality environment, identifying scanner room occupancy is challenging without interfacing with HL7 feed or integrating with multi-vendor radiology information system (RIS) or health information system (HIS) applications. Scanner room occupancy and scanning progress updates would help identify time left in the exam and improve usage of scanners, scheduling of staff and resources, and aid other workflow optimization solutions within applications such as Radiology Operations Command Center (ROCC), which is a cloud-based platform.

Currently, patient occupancy within a scanning room is determined by processing video streams from cameras mounted inside or outside scanner rooms using an edge device (such as tablet PC) that requires a substantial image or video processing capability with high computational power, which adds significantly to the hardware costs. Processing images on the cloud is a viable alternative to the on-premise processing; however, transferring images of patients or staff members to the cloud may violate General Data Protection Regulation (GDPR) privacy regulations.

### SUMMARY OF THE INVENTION

Thus, there may be a need to provide an improved approach for room/patient occupancy detection.

The object of the present invention is solved by the subject-matter of the independent claims, wherein further embodiments are incorporated in the dependent claims. It should be noted that the following described aspects of the invention apply also for the apparatus and the method for processing data acquired from an area in a healthcare facility, for transfer to a cloud-based processing system for processing at the cloud-based processing system, the cloud-based processing system, the system, the computer program product, and the computer-readable medium.

According to a first aspect of the present disclosure, there is provided an apparatus for processing data acquired from an area in a healthcare facility, for transfer to a cloud-based processing system for processing at the cloud-based processing system. The apparatus comprises an input unit, a point cloud data generator, and an output unit. The input unit is configured to receive the data acquired from the area in the healthcare facility that is to be sent to the cloud-based processing system. The acquired data comprises one or more of video data, image data, and ranging data. The point cloud data generator is configured to process the acquired data to generate point cloud data usable to provide a point cloud rendering of the area in the healthcare facility. The output unit is configured to communicate with a network to transfer the generated point cloud data and corresponding metadata to the cloud-based processing system. The generated point cloud data is preferably usable for determining a presence, a location, and/or an activity of (i) one or more persons who are present in the area in the healthcare facility and/or (ii) one or more objects which are present in the area in the healthcare facility.

Accordingly, a solution architecture is proposed that makes use of a sensor arrangement to provide sensor data, such as video data, image data, and/or ranging data. The sensor data is converted to point cloud data by the point cloud data generator. The simplified data is transmitted to a remote location, i.e., the cloud-based processing system, where presence, location, and/or activities of people and/or objects may be determined from this rendering.

The approach as described herein is designed to preserve patient and staff privacy and could be used for applications such as ROCC. In addition, the approach as described herein may require almost no integration with existing HIS/RIS solutions available in the hospitals, and it may work for any type of imaging modalities. Furthermore, since only the point cloud data is captured instead of the full room images or videos, a small footprint processing edge device is sufficient to capture the point cloud data and send it to the cloud to predict the insights. The present disclosure proposes an approach for e.g., room/patient occupancy detection with minimal processing capacity requirements for edge devices and major processing allocated to the cloud. The apparatus as described herein may be embodied as, or in, the existing edge device at the healthcare facility, thereby requiring no additional devices such as tablets, Personal Computers (PCs), and servers at the hospital facility.

According to an embodiment of the present disclosure, the area in the healthcare facility comprises one or more of an imaging room, a therapy room, a patient preparing room, a patient waiting room, and a patient transfer room.

According to an embodiment of the present disclosure, the apparatus further comprises a point cloud data extractor. The point cloud data extractor is configured to extract, from the generated point cloud data, a subset of data that is usable for determining a presence, a location, and/or an activity of (i) one or more persons who are present in the area in the health care facility, and/or (ii) one or more objects which are present in the area in the healthcare facility, and to provide the extracted point cloud data to the cloud-based processing system.

Accordingly, the apparatus according to this embodiment may extract and transfer a relevant subset of data to the cloud where appropriate cloud services (e.g., machine learning services) are applied to arrive at an inference. As an example, point cloud information for the skeleton information may be only extracted and sent to the cloud for further processing, thereby providing a more efficient data transmission.

This will be described in detail hereinafter and in particular with respect to the point cloud data extractor shown in FIG. 3.

According to an embodiment of the present disclosure, the extracted subset of data comprises one or more of: point cloud data of one or more persons who are present in the imaging modality room, point cloud data of one or more objects which are present in the imaging modality room, one or more data points that have a change of coordinates, and one or more data points that are members of a defined class of points.

This will be described in detail hereinafter and in particular with respect to the point cloud data extractor shown in FIG. 3.

According to an embodiment of the present disclosure, the one or more objects comprise one or more of a scanner table, a wearable device, a mobile/movable, and an attachable device.

According to an embodiment of the present disclosure, the point cloud data extractor is configured to adjust one or more of a resolution, a size, and a sampling rate of the generated point cloud data based on a stage of a clinical workflow, and to provide the adjusted point cloud data to the cloud-based processing system.

Accordingly, the apparatus according to this embodiment may allow for dynamic adaptation of transferred and processed data to account for different stages of a clinical workflow.

This will be described in detail hereinafter and in particular with respect to the point cloud data extractor shown in FIG. 3.

According to an embodiment of the present disclosure, the point cloud data extractor is configured to adjust one or more of a resolution, a size, and a sampling rate of the generated point cloud data based on a processing requirement at the cloud-based processing system, and to provide the adjusted point cloud data to the cloud-based processing system.

Accordingly, the apparatus according to this embodiment may allow for dynamic adaptation of transferred and processed data to account for different use cases (e.g., fast versus detailed status detection).

This will be described in detail hereinafter and in particular with respect to the point cloud data extractor shown in FIG. 3.

According to a second aspect of the present disclosure, there is provided a cloud-based processing system. The cloud-based processing system comprises at least one communications interface and an analytics engine. The at least one communications interface is configured to communicate with a network to receive point cloud data that provides a point cloud rendering of an area in a healthcare facility. The analytics engine is configured to apply at least one data analysis algorithm to determine a presence, a location, and/or an activity of (i) one or more persons who are present in the area in the healthcare facility, and/or (ii) one or more objects which are present in the area in the healthcare facility based on the received point cloud data.

This will be explained in detail hereinafter and in particular with respect to the example shown in FIG. 4.

According to an embodiment of the present disclosure, the at least one data analysis algorithm comprises a self-learning algorithm configured to keep learning based on one or more of the received point cloud data, workflow data, device logging data, workflow related data, and user feedback.

According to an embodiment of the present disclosure, the analytics engine is configured to apply (i) the at least one data analysis algorithm or (ii) a rule-based model and/or a symmetry-based model to identify and fill missing data.

According to an embodiment of the present disclosure, the cloud-based processing system further comprises a data storage that is configured to store the received point cloud data and metadata. According to a third aspect of the present disclosure, there is provided a system. The system comprises a sensor arrangement comprising one or more sensors configured to acquire one or more of video data, image data, and ranging data from an area in a healthcare facility, the apparatus according to the first aspect of the present disclosure and any associated example, and the cloud-based processing system according to the second aspect of the present disclosure and any associated example.

This will be explained in detail hereinafter and in particular with respect to the exemplary systems shown in FIGS. 1, 2 and 5.

According to a fourth aspect of the present invention, there is provided a method for processing data acquired from an area in a healthcare facility, for transfer to a cloud-based processing system for processing at the cloud-based processing system, the method comprising:
receiving the data acquired from the area in the healthcare facility that is to be sent to the cloud-based processing system, wherein the acquired data comprises one or more of video data, image data, and ranging data;
processing the acquired data to generate point cloud data usable to provide a point cloud rendering of the area in the healthcare facility; and
communicating with a network to transfer the generated point cloud data and corresponding metadata to the cloud-based processing system, wherein the generated point cloud data is preferably usable for determining a presence, a location, and/or an activity of (i) one or more persons who are present in the area in the healthcare facility, and/or (ii) one or more objects which are present in the area in the healthcare facility.

This will be explained in detail hereinafter and in particular with respect to the exemplary method shown in FIG. 6.

According to a further aspect of the present disclosure, there is provided a computer program product comprising instructions which, when the program is executed by a processing unit, cause the processing unit to carry out the steps of the method according to the third aspect of the present disclosure and any associated example.

According to another aspect of the present disclosure, there is provided a computer-readable medium having stored thereon the computer program product.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

These and other aspects of the invention will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 schematically illustrates an exemplary environment that may be suitable for implementation of the present approach.

FIG. 2 schematically illustrates a further exemplary environment that may be suitable for implementation of the present approach.

FIG. 3 schematically illustrates an example of components that may be present in the apparatus.

FIG. 4 schematically illustrates an example of components that may be present in the cloud-based processing system.

FIG. 5 illustrates an example of a multi-vendor and multi-modality environment that may be suitable for implementation of the present approach.

FIG. 6 illustrates a flowchart describing a method for processing data acquired from an area in a healthcare facility, for transfer to a cloud-based processing system for processing at the cloud-based processing system.

### DETAILED DESCRIPTION OF EMBODIMENTS

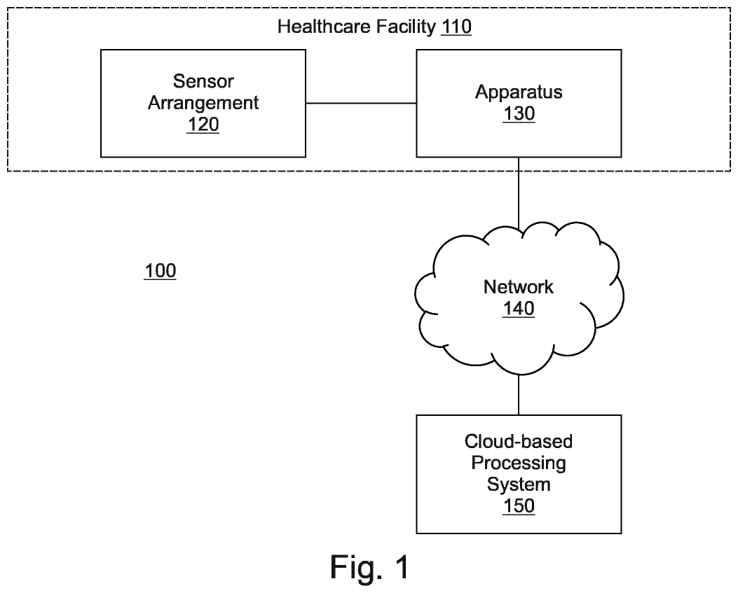

FIG. 1 schematically illustrates an exemplary environment 100 that may be suitable for implementation of the present approach. The exemplary environment 100 comprises a sensor arrangement 120, an apparatus 130, a network 140, and a cloud-based processing system 150. In the illustrated example, the sensor arrangement 120 and the apparatus 130 are both located within a healthcare facility 110. The healthcare facility 110 may be any location where healthcare is provided. Examples of the healthcare facility include, inter alia, small clinics doctor's offices, urgent care centers, and large hospitals with elaborate emergency rooms and trauma centers.

The sensor arrangement 110 of the illustrated example may include one or more sensors, which may be one or more devices, modules, or subsystems whose purpose is to detect activities, events, and/or changes in the environment at one or more locations of a healthcare facility 110, and send the acquired sensor data to some other devices, modules, subsystems, etc. The acquired sensor data may include one or more of video data, image data, and ranging data. Examples of such sensors include, inter alia, image capture devices (e.g., cameras), light detection and ranging (LiDAR) sensors, depth sensors, optical light sensors, and the like. The sensor data is not limited to the visible spectral range. Examples of the sensor data include, inter alia, grayscale images, near infrared (NIR) images, RGB images, multispectral images, hyperspectral images, and the like. The ranging data may be acquired using e.g., a stereo or multiple-camera setup system, a standard time-of-flight (TOF) technique, or any other suitable techniques.

The apparatus 130 of the illustrated example, in general, may comprise various physical and/or logical components for communicating and manipulating information, which may be implemented as hardware components (e.g. computing devices, processors, logic devices), executable computer program instructions (e.g. firmware, software) to be executed by various hardware components, or any combination thereof, as desired for a given set of design parameters or performance constraints. In some implementations, the apparatus 130 may comprise one or more microprocessors or computer processors, which execute appropriate software. The software may have been downloaded and/or stored in a corresponding memory, e.g. a volatile memory such as RAM or a non-volatile memory such as flash. The software may comprise instructions configuring the one or more processors to perform the functions described herein. It is noted that the apparatus 130 may be implemented with or without employing a processor, and also may be implemented as a combination of dedicated hardware to perform some functions and a processor (e.g. one or more programmed microprocessors and associated circuitry) to perform other functions. For example, the at least one processing unit may be implemented in the device or apparatus in the form of programmable logic, e.g. as a Field-Programmable Gate Array (FPGA).

In some examples, as shown in FIG. 1, the apparatus 130 may be embodied as, or in, an edge device. The term “edge device” at least in some examples refers a compute node that performs edge computing operations. In some examples, an edge device may be referred to as an “edge node” or “edge system”, whether in operation as a client, server, or intermediate entity. In some examples, the apparatus 130 may be incorporated into a server, gateway, on premise unit, or end consuming device, or the like.

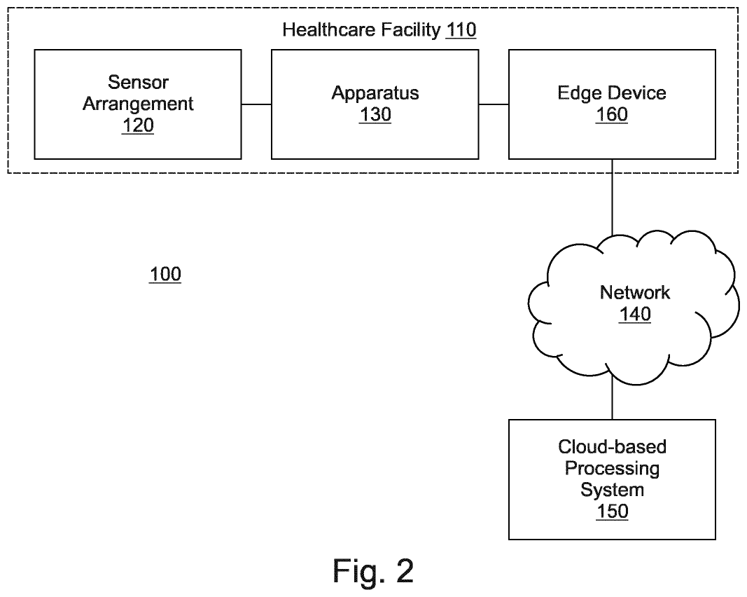

In some other examples, as shown in FIG. 2, the apparatus 130 may be embodied as a device that is coupled to an edge device 160 that has one or more communications interfaces configured to transmits data to and receive data from the cloud-based processing system 150 over the network 140. Although the apparatus 130 is illustrated as a hardware system in FIGS. 1 and 2 by way of example, it will be appreciated that in alternative embodiments, the apparatus 130 may be embodied as a software system (e.g., a software residing in an edge device) that directs hardware to perform the operations.

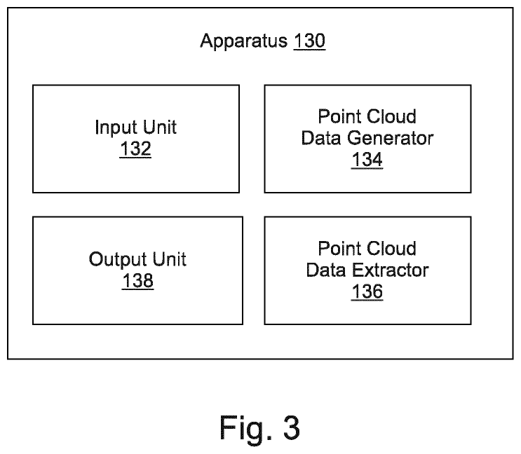

The apparatus 130 is described in greater detail with respect to an example shown in FIG. 3.

The network 140 may represent a network such as the Internet, a wireless local area network (WLAN), or a wireless wide area network (WWAN) including proprietary and/or enterprise networks for a company or organization, a cellular core network (e.g., an evolved packet core (EPC) network, a NextGen Packet Core (NPC) network, a 5G core (5GC), or some other type of core network), a cloud computing architecture/platform that provides one or more cloud computing services, and/or combinations thereof. As examples, the network 140 and/or access technologies may include cellular technology such as LTE.

The cloud-based processing system 150 may represent one or more application servers, a cloud computing architecture/platform that provides computing services, and/or some other remote infrastructure. The cloud computing services (also referred to as cloud services) are one or more capabilities offered via cloud computing that are invoked using a defined interface (e.g., an API or the like). The cloud-based processing system 150 may include any one of a number of services and capabilities, such as occupancy detection (e.g., general availability of the room or momentary presence and quantity of persons, etc.) and more detailed analysis (e.g., number and interaction of persons, patient dimensions and contour, etc.).

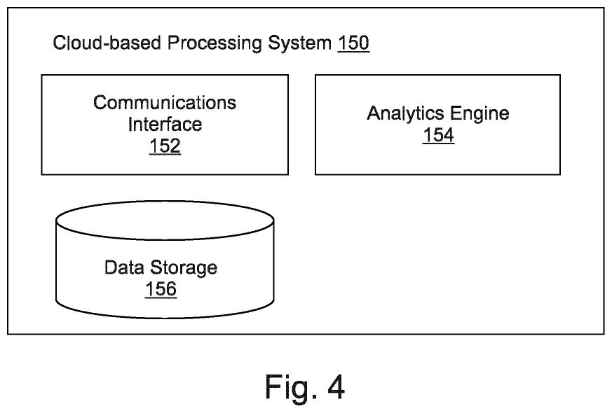

The cloud-based processing system 150 is described in greater detail with respect to the example shown in FIG. 4 FIG. 3 illustrates an example of components that may be present in the apparatus 130 for implementing the techniques described herein. The apparatus 130 may include any combinations of the hardware or logical components referenced herein, and it may include or couple with any device usable with a communication network or a combination of such networks. The components may be implemented as ICs, portions thereof, discrete electronic devices, or other modules, instruction sets, programmable logic or algorithms, hardware, hardware accelerators, software, firmware, or a combination thereof adapted in the apparatus 130, or as components otherwise incorporated within a chassis of a larger system.

The apparatus 130 of the illustrated example includes an input unit 132, a point cloud data generator 134, a point cloud data extractor 136, and an output unit 138. In alternative configurations, different and/or additional components may be included in the apparatus 130. Additionally, functionality described in conjunction with one or more of the components shown in FIG. 3 may be distributed among the components in a different manner than described in conjunction with FIG. 3 in some examples.

The input and output units 132 and 138 may be embodied as circuitry and/or components to facilitate input/output operations with the point cloud data generator 134 and the point cloud data extractor 136. For example, the input unit 132 and the output unit 138 may be embodied as, or otherwise include, memory controller hubs, input/output control hubs, integrated sensor hubs, firmware devices, communication links (e.g., point-to-point links, bus links, wires, cables, light guides, printed circuit board traces, etc.), and/or other components and subsystems to facilitate the input/output operations. In some examples, the input unit 132 and the output unit 138 may form a portion of an SoC and be incorporated into the compute subsystem.

The point cloud data generator 134 and the point cloud data extractor 136 may be embodied as an integrated circuit, an embedded system, an FPGA, a System-on-Chip (SoC), or other integrated system or device. In some implementations, the point cloud data generator 134 and the point cloud data extractor 136 include or are embodied as a processor and a memory. The processor may be embodied as any type of processor capable of performing the functions described herein (e.g., executing an application). For example, the processor may be embodied as a multi-core processor(s), a microcontroller, or other processor or processing/controlling circuit. The processor may be embodied as, include, or be coupled to an FPGA, an application specific integrated circuit (ASIC), reconfigurable hardware or hardware circuitry, or other specialized hardware to facilitate performance of the functions described herein.

In some examples, the output unit 138 may include or be coupled to a communications circuitry, which may be embodied as any communication circuit, device, or collection thereof, capable of enabling communications over a network between the apparatus 130 and the cloud-based processing system 150. The communication circuitry may be configured to use any one or more communication technology (e.g., wired or wireless communications) and associated protocols (e.g., a cellular networking protocol such a 3GPP 4G or 5G standard, a wireless local area network protocol such as IEEE 802.11/WiFi®, a wireless wide area network protocol, Ethernet, Bluetooth®, Bluetooth Low Energy, a IoT protocol such as IEEE 802.15.4 or ZigBee®, low-power wide-area network (LPWAN) or low-power wide-area (LPWA) protocols, etc.) to effect such communication.

In use, the apparatus 130 receives, via the input unit 132, sensor data acquired by the sensor arrangement 110 which is usable for detecting activities, events, and/or changes in the environment at one or more locations of a healthcare facility 110. The sensor data may include video data, image data, and/or ranging data.

The point cloud data generator 134 processes the acquired sensor data to generate point cloud data and corresponding metadata usable to provide a point cloud rendering of the area in the healthcare facility. The point cloud data consists of a collection of points called a point cloud. The point cloud comprises a set of individual three-dimensional points. Each point, in addition to having a three-dimensional (x, y, z) position may also contain a number of further attributes such as color, reflectance, surface normal, etc. The metadata may comprise additional information about the point cloud data, e.g., reliability of point coordinates, tracking vector which is dependent on underlying point cloud generation technique, and the like.

For example, the sensor arrangement 110 may comprise multiple video or imaging cameras that arranged to capture color attributes in the scene at one or more locations in the healthcare facility. The location of the objects and/or people can be captured using various approaches including, inter alia, infrared depth cameras, photogrammetry and stereo disparity, and illumination of the scene with structured light or lasers. The acquired sensor data is then processed using an algorithm to obtain a sparse vowelized point cloud representing the capture scene. An example of a method for capturing voxelized point clouds using only cameras is described in C. Loop, C. Zhang and Z. Zhang, “Real-time high-resolution sparse voxelization with application to image-based modeling”, Proc. 5th High-Perform. Graph. Conf., pp. 73-79, 2013.

As an option, the point cloud data generated by the point cloud data generator 134 may be provided as an input to the point cloud data extractor 136, which is configured to extract, from the generated point cloud data, a subset of data that is usable for determining a presence, a location, and/or an activity of one or more persons who are present in the area in the health care facility and to provide the extracted point cloud data to the cloud-based processing system. Alternatively or additionally, the point cloud data extractor 136 may be configured to extract, from the generated point cloud data, a subset of data that is usable for determining a presence, a location, and/or an activity of one or more objects which are present in the area in the healthcare facility and to provide the extracted point cloud data to the cloud-based processing system.

The extracted subset of data may comprise one or more of (i) point cloud data of one or more persons who are present in the imaging modality room, (ii) point cloud data of one or more objects which are present in the imaging modality room, (iii) one or more data points that have a change of coordinates, and (iv) one or more data points that are members of a defined class of points.

For example, the point cloud data extractor 136 may extract a subset of points from the generated point cloud based on the indices output by a segmentation algorithm, which associates each pixel or voxel in the acquired video, image, and/or ranging data with a class label, such as person (e.g., patient), or object (e.g., scanner table, wearable device, mobile/movable, attachable device, etc.). Examples of such segmentation algorithm include, inter alia, U-Net, DeepLab, Convolutional Neural Network (CNN), and the like. As an example, the extracted subset of points may comprise only skeleton information. The skeleton of a shape, especially an articulated shape such as a human or an object, provides an intuitive and effective abstraction which facilitates determining a presence, a location, and/or an activity of one or more persons and/or objects that are present at one or more locations in the healthcare facility.

In some examples, the sensor arrangement may comprise one or more cameras that support Software Development Kits (SDKs). Using the SDKs supported by the cameras, the point cloud data extractor 134 may only extract the point cloud information for the skeleton information from the generated point cloud.

In some examples, the point cloud data extractor 136 may extract a subset of points representing a boundary of a scanner table from the point cloud data generated by the point cloud data generator 134 so that the table motion can be inferred. In some example, the point cloud data extractor 136 may extract a subset of points representing e.g., a wearable device, a mobile/movable, and an attachable device (e.g., coils) from the point cloud data generated by the point cloud data generator 134 so that the patient motion can be inferred.

Such simple criterion could be transmitting only those data points where there is a change in the coordinates. In other words, the point cloud data extractor 136 in some examples may extract a subset of points representing one or more data points that have a change of coordinates from the point cloud data generated by the point cloud data generator 134 so the motion of a person and/or an object can be inferred.

As a further option, the extraction of the subset, e.g., the resolution, size, and/or sampling rate of point cloud data stream, may be dynamically adjusted.

In one example, depending on the specific use-case of the occupancy data (e.g., general availability of the room or momentary presence and quantity of persons), the extraction of the subset, e.g., the resolution, size and/or sampling rate, may be adjusted. Specifically, one low resolution point cloud data stream could be used for a fast and low latency analysis, e.g., in order to get information if any body is present. One high resolution and higher latency point cloud data stream could be used for obtaining slower but more detailed analysis results, such as number and interaction of persons, patient dimensions and contour, etc.

In another example, the use and activation of these streams could be dynamically adjusted depending on the stage of the clinical workflow and/or other available information. For example, when nobody is present or moving in the scene detected by the sensor arrangement, one low resolution point cloud data stream could be transferred to the cloud-based processing system. On the other hand, when it is required to generate patient contour to guide patient positioning while on the table, one high resolution and higher latency point cloud data stream could be transferred to the cloud-based processing system for a slower but more detailed analysis.

FIG. 4 illustrates an example of components that may be present in the cloud-based processing system 150 for implementing the techniques described herein. The cloud-based processing system 150 may include any combinations of the hardware or logical components referenced herein, and it may include or couple with any device usable with a communication network or a combination of such networks.

The cloud-based processing system 150 of the illustrated example comprises a communications interface 152, an analytics engine 154, and a data storage 156.

The communications interface 152 may be a hardware element, or collection of hardware elements, used to communicate over one or more networks (e.g., network 140) and/or with other devices (e.g., apparatus 130 or edge device 160). The communications interface 152 may be configured to use any one or more communication technology (e.g., wired or wireless communications) and associated protocols (e.g., a cellular networking protocol such a 3GPP 4G or 5G standard, a wireless local area network protocol such as IEEE 802.11/WiFi®, a wireless wide area network protocol, Ethernet, Bluetooth ®, Bluetooth Low Energy, a IoT protocol such as IEEE 802.15.4 or ZigBee®, low-power wide-area network (LPWAN) or low-power wide-area (LPWA) protocols, etc.) to effect such communication. The analytics engine 154 provides one or more cloud computing services to analyse the point cloud data received from the apparatus 130 and transfer the analysis results to the apparatus 130 via the communications interface 152. Examples of such services include, inter alia, patient occupancy analysis, room availability analysis, activity detection, etc. The cloud computing services provided by the analytics engine 154 may include machine learning (ML) services, and/or other like services for performing various analysis. The term “machine learning” or “ML” at least in some examples refers to the use of computer systems implementing algorithms and/or statistical models to perform specific task(s) without using explicit instructions, but instead relying on patterns and inferences. ML algorithms build or estimate mathematical model(s) (referred to as “ML models” or the like) based on sample data (referred to as “training data,” “model training information,” or the like) in order to make predictions or decisions without being explicitly programmed to perform such tasks. Generally, an ML algorithm is a computer program that learns from experience with respect to some task and some performance measure, and an ML model may be any object or data structure created after an ML algorithm is trained with one or more training datasets. After training, an ML model may be used to make predictions on new datasets. Although the term “ML algorithm” at least in some embodiments refers to different concepts than the term “ML model,” these terms as discussed herein may be used interchangeably for the purposes of the present disclosure.

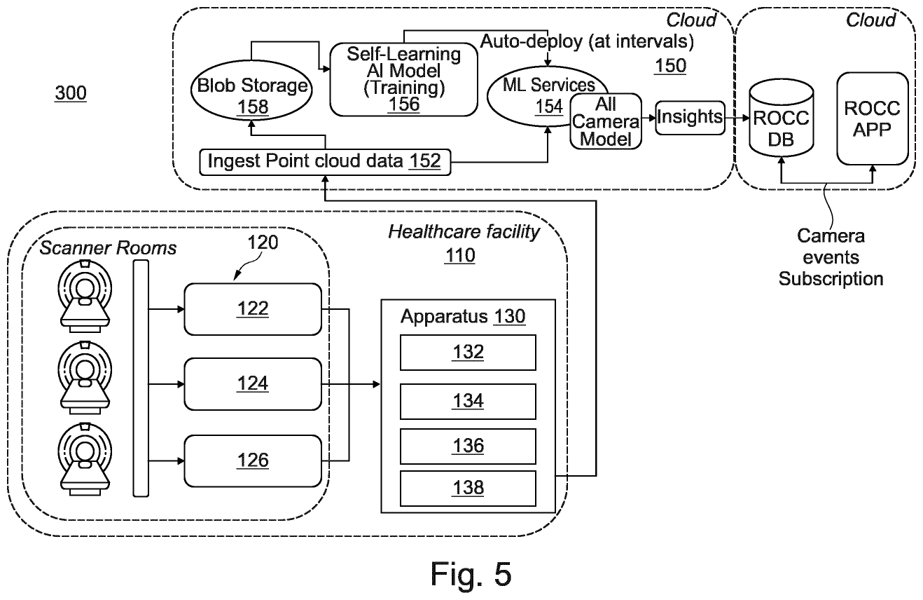

In some examples, a self-learning ML model may be deployed into one or more MR services. For example, the data storage 156 may store the point cloud data received from the apparatus 130 in a blob type of storage. From the data storage 156, the point cloud data is fed into the self-learning model, which is regularly deployed into ML services. This solution may support self-learning AI model component which keeps learning based on the input point cloud data, which comes from different modalities, different hospital facilities and it keeps learning to strengthen its model and provide more reliable and robust results. An example of the deployment of a self-learning ML model is illustrated in FIG. 5.

Apart from this, these trained ML models may identify and auto-fill missing data resulting from occlusions, truncated objects while extracting the point cloud data. Alternatively, these ML models could be replaced by rule-based and symmetry-based models for auto-filling the missing data.

FIG. 5 illustrates an example of a multi-vendor and multi-modality environment 300 that may be suitable for implementation of the present approach. The exemplary environment 300 comprises a sensor arrangement 120, an apparatus 130, and a cloud-based processing system 150. Although only one apparatus 130 is illustrated in FIG. 5 by way of example, it will be appreciated that in alternative embodiments, two or more apparatuses 130 may be provided within the healthcare facility 110.

In the illustrated example, the environment 300 comprises one or more scanner room. In each scanner room, there is one or more medical imaging modalities to generate medical imaging data for a patient. Although three medical imaging modalities are illustrated in FIG. 5 by way of example, it is to be appreciated that lesser or more medical imaging modalities than the example described herein may be used for certain implementations. Examples of the medical imaging modalities may include, but are not limited to, Magnetic Resonance Imaging (MRI), ultrasound, medical radiation, angiography, and Computed Tomography (CT) scanners. The medical imaging modalities may be used in fields including, but not limited to, radiology, cardiology, oncology, nuclear medicine, radiotherapy, neurology, orthopedics, obstetrics, gynecology, ophthalmology, dentistry, maxillofacial surgery, dermatology, pathology, clinical trials, veterinary medicine, and medical/clinical photography.

The sensor arrangement 120 is used to record activities, events, and/or changes within the one or more scanner rooms, and to provide sensor data which comprises the information about the detected activities, events, and/or changes to an apparatus 130. The sensor arrangement may comprise one or more sensors, such as sensors 122, 124, and 126 shown in FIG. 5, which may be mounted inside or outside the scanner rooms. Although three sensors 122, 124, and 126 are illustrated in FIG. 5 by way of example, it is to be appreciated that lesser or more sensors than the example described herein may be used for certain implementations. The sensor data comprises video data, image data, and/or ranging data. In some examples, the sensor arrangement may comprise one or more sensors that have in-built capability of algorithms to detect objects and support the depth sensing as well.

The apparatus 130 of the illustrated example is embodied as, or in, an edge device, which sends data to or receives data from the cloud-based processing system 150. The apparatus 130 comprises a sensor data interface 132, a point cloud data generator 134, a point cloud data extractor 136, and a communications interface 136.

The sensor data interface 132 may include one or more physical ports, such as USB, Bluetooth, Ethernet, wireless Ethernet, etc., for communicating with the sensor arrangement 110 to receive the acquired sensor data.

The point cloud data generator 134 processes the acquired data to generate point cloud data usable to provide a point cloud rendering of the area in the healthcare facility. Since only the point cloud data is captured instead of the full room images and/or videos, a small footprint processing edge device is sufficient to capture the point cloud data and send it to the cloud-based processing system 150 to e.g., build the ML self-learning models to predict the insights.

In some examples, only a subset of the point cloud data is sent to the cloud-based processing system 150 which is sufficient to e.g., build the ML self-learning models to predict the insights. In such examples, the point cloud data extractor 136 may extract the relevant subset from the point cloud data generated from the point cloud data generator 134. As an example, features extracted from point cloud may be used to identify patients, staff members and other persons, with data sent to the cloud-based processing system 150 for further processing. As a further example, using the SDKs supported by the cameras the point cloud information for the skeleton information may be only extracted and sent to the cloud-based processing system 150 for further processing. The idea of extracting only the point cloud with respect to skeleton images is to avoid transferring the less relevant data to cloud, e.g., not sending the point cloud data of the whole scene that requires a lot of processing and data transfer as well to the cloud-based processing system. Instead send a few key points, for example, boundary of the scanner table so that table motion can be inferred. Same applies to coils as well. In addition to this, there are other approaches aimed at reducing data transfer to cloud. For example, a simple criterion could be transmitting only those data points where there is a change in the coordinates. Such approaches could potentially improve the efficiency and latency of the solution.

In some examples, the extraction of the subset, e.g., the resolution, size, and/or sampling rate may be adjusted in dependence on the specific use-case of the occupancy data. For example, one low resolution point cloud data stream may be used for a fast and low latency analysis, e.g., to get information if anybody is present, whereas one high resolution point cloud data stream may be used for a slower and high latency analysis with more detailed analysis results (e.g., number and interaction of persons, patient dimensions and contour). The use and activation of these streams may be dynamically adjusted depending on the stage of the clinical workflow and/or other available information. For example, a high resolution point cloud data stream may be used in order to generate patient contour to guide patient positioning while on the table.

Via the communications interface 138, the point cloud data is then sent to the cloud-based processing system 150 for further processing.

The cloud-processing system 150 comprises a communications interface 152, an analytics engine 154, and a data storage 156. The communications interface 152 is configured to communicate with a network to receive the point cloud data from the apparatus 130. The data storage 156 may store the point cloud data in a blob type of storage. From the data storage 156 the point cloud data may be fed into a self-learning model provided by the analytics engine 154, which is regularly deployed into ML services. At regular intervals, these self-supervised models will be evaluated and deployed at one or more ML services to achieve performance gains. These ML services may include a variety of cloud services, such as room availability detection, patient occupancy detection, etc., using the existing models. Apart from this, these trained ML models may identify and auto-fill missing data resulting from occlusions, truncated objects while extracting the point cloud data. Alternatively, these ML models could be replaced by rule-based and symmetry-based models for auto-filling the missing data. Activity detection based on point cloud data is one potential use case for self-supervised learning. Identifying actions detected individuals are involved in could help clarify their roles (e.g., patient versus staff member) and help identify stage is the workflow taking place within the scanner room.

The predicted insights information will be used by ROCC application for users to view and plan for further scheduling of rooms, staff, and other resources, etc.

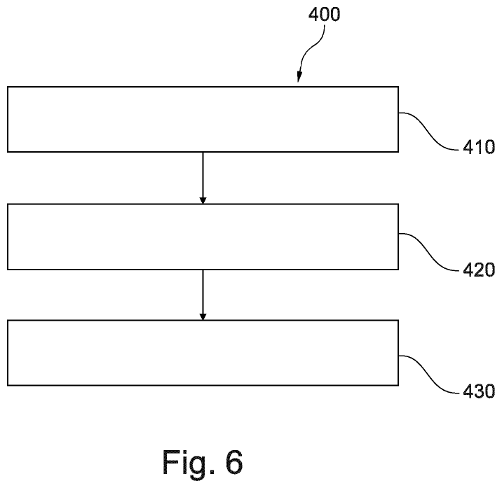

FIG. 6 illustrates a flowchart describing a method 400 for processing data acquired from an area in a healthcare facility, for transfer to a cloud-based processing system for processing at the cloud-based processing system. The method may be used in conjunction with the other methods and systems described herein. In particular, the method 400 shown in FIG. 6 may be implemented on the exemplary environments shown in FIGS. 1, 2 and 5.

The method may be at least partly computer-implemented, and may be implemented in software or in hardware, or in software and hardware. Further, the method may be carried out by computer program instructions running on means that provide data processing functions. The data processing means may be a suitable computing means, such as an electronic control module etc., which may also be a distributed computer system. The data processing means or the computer, respectively, may comprise of one or more processors, a memory, a data interface, or the like.

At block 410, the method comprises a step of receiving the data acquired from the area in the healthcare facility that is to be sent to the cloud-based processing system. The acquired data comprises one or more of video data, image data, and ranging data. When the method 200 is implemented on the apparatus shown in FIG. 3, block 410 may be implemented using the data interface 132 that is communicatively coupled to the sensor arrangement to receive the acquired sensor data.

At block 420, the method comprises a step of processing the acquired data to generate point cloud data usable to provide a point cloud rendering of the area in the healthcare facility. This may be implemented by the point cloud data generator 134 shown in FIG. 3.

Optionally, only a subset of the point cloud data is sent to the cloud-based processing system 150 for further processing in order to avoid transferring less relevant data to cloud, because sending the point could data of the whole scene and video may require a lot of processing and data transfer as well to the cloud-based processing system 150. When the method 200 is implemented on the apparatus shown in FIG. 3, this optional step may be implemented using the point cloud data extractor 136.

At block 430, the method comprises the step of communicating with a network to transfer the generated point cloud data and corresponding metadata to the cloud-based processing system for further processing. In some examples, the generated point cloud data is preferably usable for determining a presence, a location, and/or an activity of one or more persons who are present in the area in the healthcare facility. Alternatively or additionally, the generated point cloud data is preferably usable for determining one or more objects which are present in the area in the healthcare facility. This may be implemented by the output unit 138 shown in FIG. 3, which may include or coupled to a communications interface to send data to and receive data from the cloud-based processing system 150.

In another exemplary embodiment of the present invention, a computer program or a computer program element is provided that is characterized by being adapted to execute the method steps of the method according to one of the preceding embodiments, on an appropriate system.

The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfil the procedure of an exemplary embodiment of the method as described above.

According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

In the claims, the word “comprising” does not exclude other elements or steps, and the indefinite article “a” or “an” does not exclude a plurality.

1. An apparatus for processing data acquired from an area in a healthcare facility for transfer to a cloud-based processing system, the apparatus comprising:
a processor operatively coupled to memory, the processor configured to:
receive the data acquired from the area in the healthcare facility that is to be sent to the cloud-based processing system, wherein the acquired data comprises one or more of video data, image data, and ranging data;
process the acquired data to generate point cloud data usable to provide a point cloud rendering of the area in the healthcare facility; and
communicate with a network to transfer the generated point cloud data and corresponding metadata to the cloud-based processing system, wherein the generated point cloud data is used for determining at least one of a presence, a location, and an activity of at least one of (i) one or more persons who are present in the area in the healthcare facility and (ii) one or more objects which are present in the area in the healthcare facility.

2. The apparatus according to claim 1,
wherein the area in the healthcare facility comprises one or more of:
an imaging room;
a therapy room;
a patient preparing room;
a patient waiting room; and
a patient transfer room.

3. The apparatus according to claim 1, wherein the processor is further configured to:
extract, from the generated point cloud data, a subset of data that is used for determining the at least one of a presence, a location, and/or an activity of the at least one of (i) one or more persons who are present in the area in the health care facility, and (ii) one or more objects which are present in the area in the healthcare facility, and
provide the extracted point cloud data to the cloud-based processing system.

4. The apparatus according to claim 3,
wherein the extracted subset of data comprises one or more of:
point cloud data of one or more persons who are present in the imaging modality room;
point cloud data of one or more objects which are present in the imaging modality room;
one or more data points that have a change of coordinates; and
one or more data points that are members of a defined class of points.

5. The apparatus according to claim 4,
wherein the one or more objects comprise one or more of a scanner table, a wearable device, a mobile/movable device, and an attachable device.

6. The apparatus according to claim 3,
wherein the processor is further configured to:
adjust one or more of a resolution, a size, and a sampling rate of the generated point cloud data based on a stage of a clinical workflow, and
provide the adjusted point cloud data to the cloud-based processing system.

7. The apparatus according to claim 3,
wherein the processor is further configured to:
adjust one or more of a resolution, a size, and a sampling rate of the generated point cloud data based on a processing requirement at the cloud-based processing system; and
provide the adjusted point cloud data to the cloud-based processing system.

8. The apparatus according to claim 1, further comprising:
at least one communications interface configured to communicate with the network to receive the point cloud data that provides the point cloud rendering of the area in a healthcare facility; and
wherein the processor is further configured to apply at least one data analysis algorithm to determine the at least one of a presence, a location, and an activity of the at least one of (i) one or more persons who are present in the area in the healthcare facility, and (ii) one or more objects which are present in the area in the healthcare facility based on the received point cloud data.

9. The apparatus according to claim 8,
wherein the at least one data analysis algorithm comprises a self-learning algorithm configured to keep learning based on one or more of the received point cloud data, workflow data, device logging data, workflow related data, and user feedback.

10. The apparatus according to claim 8,
wherein the processor is further configured to apply (i) the at least one data analysis algorithm or (ii) at least one of a rule-based model and a symmetry-based model to identify and fill missing data.

11. The apparatus according to claim 8, further comprising:
a data storage configured to store the received point cloud data and metadata.

12. The apparatus according to claim 8, further comprising:
a sensor arrangement comprising one or more sensors configured to acquire one or more of video data, image data, and ranging data from an area in a healthcare facility.

13. A method for processing data acquired from an area in a healthcare facility for transfer to a cloud-based processing system, the method comprising:
receiving the data acquired from the area in the healthcare facility that is to be sent to the cloud-based processing system, wherein the acquired data comprises one or more of video data, image data, and ranging data;
processing the acquired data to generate point cloud data usable to provide a point cloud rendering of the area in the healthcare facility; and
communicating with a network to transfer the generated point cloud data and corresponding metadata to the cloud-based processing system, wherein the generated point cloud data is used for determining at least one of a presence, a location, and an activity of at least one of (i) one or more persons who are present in the area in the healthcare facility, and (ii) one or more objects which are present in the area in the healthcare facility.

14. (canceled)

15. A non-transitory computer-readable medium having stored thereon instruction for processing data acquired from an area in a healthcare facility for transfer to a cloud-based processing system, the instructions, when executed by a processor, cause the processor to:
receive the data acquired from the area in the healthcare facility that is to be sent to the cloud-based processing system, wherein the acquired data comprises one or more of video data, image data, and ranging data;
process the acquired data to generate point cloud data usable to provide a point cloud rendering of the area in the healthcare facility; and
communicate with a network to transfer the generated point cloud data and corresponding metadata to the cloud-based processing system, wherein the generated point cloud data is used for determining at least one of a presence, a location, and an activity of at least one of (i) one or more persons who are present in the area in the healthcare facility and (ii) one or more objects which are present in the area in the healthcare facility.

16. The method according to claim 13, further comprising:
applying at least one data analysis algorithm to determine the at least one of a presence, a location, and an activity of the at least one of (i) one or more persons who are present in the area in the healthcare facility, and (ii) one or more objects which are present in the area in the healthcare facility based on the received point cloud data.

17. The method according to claim 16, further comprising:
applying (i) the at least one data analysis algorithm or (ii) at least one of a rule-based model and a symmetry-based model to identify and fill missing data.

18. The method according to claim 13, further comprising:
adjusting one or more of a resolution, a size, and a sampling rate of the generated point cloud data based on at least one of a stage of a clinical workflow and a processing requirement at the cloud-based processing system; and
providing the adjusted point cloud data to the cloud-based processing system.

19. The non-transitory computer-readable medium according to claim 15, wherein the instructions, when executed by the processor, further cause the processor to:
apply at least one data analysis algorithm to determine the at least one of a presence, a location, and an activity of the at least one of (i) one or more persons who are present in the area in the healthcare facility, and (ii) one or more objects which are present in the area in the healthcare facility based on the received point cloud data.

20. The non-transitory computer-readable medium according to claim 19, wherein the instructions, when executed by the processor, further cause the processor to:
apply (i) the at least one data analysis algorithm or (ii) at least one of a rule-based model and a symmetry-based model to identify and fill missing data.

21. The non-transitory computer-readable medium according to claim 15, wherein the instructions, when executed by the processor, further cause the processor to:
adjust one or more of a resolution, a size, and a sampling rate of the generated point cloud data based on at least one of a stage of a clinical workflow and a processing requirement at the cloud-based processing system; and
provide the adjusted point cloud data to the cloud-based processing system.
